(19) United States
(12) Patent Application Publication
(10) Pub. No.: US 2008/0193543 A1
(43) Pub. Date: Aug. 14, 2008

(54) Drug Delivery Formulations For Targeted Delivery

(75) Inventors: Arthur Peter Morello, III, Pawtucket, RI (US); Joshua Reineke, Providence, RI (US); Peter Matthew Cheifetz, Burlington, MA (US); Bryan Laulicht, New Hyde Park, NY (US); Edith Mathiowitz, Brookline, MA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Brown University Research Foundation, Providence, RI (US)

(21) Appl. No.: 11/914,394

(22) PCT Filed: May 17, 2006
(86) PCT No.: PCT/US2006/019229

§ 371 Date: Nov. 14, 2007

Related U.S. Application Data
(60) Provisional application No. 60/682,213, filed on May 17, 2005.

(51) Int. Cl. A61K 9/16; A61N 2/00

(52) U.S. Cl. 424/490; 424/489; 600/9

(57) ABSTRACT

The size and location of microsphere uptake/delivery are important determinants of the final biodistribution of oral microsphere systems. Formulations, kits, methods of administering the formulations, and using the kits are described herein. The formulations are oral dosage formulations. In one embodiment, the formulations contain microparticles and/or nanoparticles having a homogenous size range selected to optimize uptake in a specific region of the GI tract and target drug delivery to specific organs. In some embodiments, the dosage formulation contains an enteric coating and/or a magnetic material. In a preferred embodiment, the formulation contains a magnetic material and an active agent to be delivered, optionally the active agent is in the form of micro- or nano-particles. In some embodiments metallomucoadhesive materials and/or magnetic materials are employed as magnetic and/or mucoadhesive sources. Formulations containing magnetic materials can be localized using the kits and methods disclosed herein. In one embodiment, the method includes orally administering the formulation and applying an extracorporeal magnet to a site on the outside surface of the patient's body in an area that closely apposes the location in the gastrointestinal tract to which delivery of the formulation is desired. The extracorporeal magnet is applied for a suitable time period to allow for the drug to be released from the formulation and/or to allow for the formulation to adhere to the site. Both magnetic and mucoadhesive forces may be utilized to site-direct and retain the dosage form in the region of the gastrointestinal (GI) tract most suitable for the desired delivery.

### CROSS-REFERENCE TO RELATED APPLICATIONS

This application claims priority to U.S. Ser. No. 60/682,213, filed May 17, 2005.

### FIELD OF THE INVENTION

The present invention is in the field of targeted delivery of active agents.

### BACKGROUND OF THE INVENTION

The mechanisms of oral microparticle and nanoparticle drug delivery are still largely unknown. In the mid-1980's, a number of important discoveries regarding drug delivery via microparticles were being made. U.S. Pat. No. 5,853,763 to Tice, et al. was the first to report the importance of size in determining specificity of uptake, with microparticles under ten microns in diameter being selectively phagocytized by the cells in the Peyer's Patches. Subsequent discoveries in pulmonary delivery have demonstrated that particles of approximately three to five microns are essential for delivery to the deep lung.

Today, improving the oral delivery of pharmaceutical compounds that have poor oral bioavailability (<1%) is the primary motivation behind using particulate-based drug delivery systems. It is known that drug-loaded particles made from polymers can cross absorptive epithelium (see e.g. Hillery, et al., Journal of Drug Targeting 2:151-156 (1994); Florence, A T., Drug Discovery Today: Technologies, 2:75-81 (2005)), potentially improving the bioavailability of many drugs. To date, the mechanisms of particle uptake by the intestinal epithelium remain unclear (Florence, A T., Drug Discovery Today: Technologies, 2:75-81 (2005)), but it is known that decreasing the particle size of drug-loaded particles facilitates their uptake throughout the alimentary canal (see Eldridge, et al., Advances in Experimental Medicine and Biology, 251:191-202 (1989)). Given the importance of size, ideally, one would fabricate particles with minimal aggregation. Particle aggregates used in oral drug delivery have poor uptake in the gastrointestinal (GI) tract, reducing the efficacy of drug-loaded microspheres (Florence, et al., Advanced Drug Delivery Reviews, 50:S69-S89 (2001)). For these reasons, it would be advantageous to use populations of particles that: (1) have a small particle size (<1 micron); (2) have minimal aggregation (coefficient of variance<25%); and (3) can selectively target specific regions of the GI tract to maximize particle uptake.

More recent studies have demonstrated that targeted uptake can be most easily obtained by binding of targeting ligands to the microparticles. This requires knowledge of the target, and of a suitable ligand, and greatly complicates regulatory review.

None of these methods target to a particular type of systemic delivery, only to types of tissues, or local or systemic delivery.

It is therefore an object of the present invention to provide a means for targeting of microparticles to specific regions in the gastrointestinal tract without the use of ligands.

It is another object of the present invention to provide a means for targeting of microparticles that is selective for different systemic vascular regions.

### BRIEF SUMMARY OF THE INVENTION

Formulations, kits, methods of administering the formulations, and using the kits are described herein. The formulations are oral dosage formulations. In one embodiment, the formulations contain microparticles and/or nanoparticles having a homogenous size range selected to optimize uptake in a specific region of the GI tract and target drug delivery to specific organs. In some embodiments, the dosage formulation contains an enteric coating and/or a magnetic material. In a preferred embodiment, the formulation contains a magnetic material and an active agent to be delivered. These formulations may contain conventional controlled release systems. Optionally, the formulation contains the active agent is in the form of micro- or nano-particles, preferably having a homogenous size range selected to optimize uptake in a specific region of the GI tract and target drug delivery. In some embodiments metallomucoadhesive materials and/or magnetic materials are employed as magnetic and/or mucoadhesive sources.

Formulations containing magnetic materials can be localized using the kits and methods disclosed herein. In one embodiment, the method includes orally administering the formulation and applying an extracorporeal magnet to a site on the outside surface of the patient's body in an area that closely apposes the location in the gastrointestinal tract to which delivery of the formulation is desired. The extracorporeal magnet is applied for a suitable time period to allow for the drug to be released from the formulation and/or to allow for the formulation to adhere to the site. Both magnetic and mucoadhesive forces may be utilized to site-direct and retain the dosage form in the region of the gastrointestinal (GI) tract most suitable for the desired delivery.

### DETAILED DESCRIPTION OF THE INVENTION

The size and location of microsphere uptake/delivery are important determinants of the final biodistribution of oral microsphere systems. The difference in biodistribution profiles is a direct effect of the microsphere size. When comparing 0.5, 1, 2, and 5 micron diameter microparticles, the amount of uptake was greatest with 0.5 μm microspheres (45.8%), less with 1 μm microspheres (28.9%) and insignificant with the 2 and 5 μm microspheres. The biodistribution of the microspheres also varied with the microsphere size. 0.5 μm microspheres isolated to the portal blood and liver, suggesting retention by the liver via a first pass mechanism. However, the larger 1 μm microspheres distributed throughout most tissues, concentrating mostly in the lung. In addition, a substantial fraction of the microspheres were still circulating in the central blood compartment after the 5 hour time period.

In addition to the size parameter, the location of microsphere delivery directly affects the biodistribution profile. 1 μm microspheres were delivered locally to either the jejunum or ileum, or orally. The jejunum delivery resulted in high delivery to the lung (25.8%). However, the ileum delivery seemed to shift the distribution to the liver. An oral gavage study of 1 μm microspheres had a much different biodistribution profile than the same microspheres delivered locally to the jejunum. The majority of the microspheres from the fed study were found in the liver and portal blood. In addition, the size dependence of uptake differed for each region suggesting separate mechanisms of uptake.

Accordingly, maximum delivery of microparticulate drug is obtained by delivery of microparticles between 0.5 and 2 microns in diameter, preferably about 1 micron in diameter. In the most preferred embodiment, the microparticles are in the form of a homogenous population of preferably 75%, more preferably 80%, most preferably about 90% or more, having a mean diameter of about one micron (majority range between 0.5 and 2 microns). This is also the preferred formulation for targeted delivery to all organs, especially the pulmonary region. The preferred location for release of the microparticles for delivery throughout the systemic circulation is the ileum.

In contrast, where the target is the portal circulation, especially the liver, it is preferable to deliver microparticles of about 0.5 microns or less in diameter, preferably about 0.5 microns in diameter. In the most preferred embodiment, the microparticles are in the form of a homogenous population of preferably 75%, more preferably 80%, most preferably about 90% or more, having a mean diameter of about 0.5 micron (majority range between 400 and 500 nm). This is also achieved through delivery of the microparticles to the jejunum rather than the ileum, which can be achieved through the use of enteric coated formulations that do not dissolve until the particles reach the jejunum.

Targeted delivery to the desired tissues or organs can be achieved through the use of formulations coated with or containing one or more magnetic materials. In a preferred embodiment the formulation contains magnetic materials and delivery is directed by applying an extracorporeal magnet to the surface of the skin in an area adjacent to the desired organ or tissue for delivery.

### I. DEFINITIONS

“Alloys” as used herein refers to a homogeneous mixture or solid solution of two or more metals, the atoms of one replacing or occupying interstitial positions between the atoms of the other.

“Dosage form” as used herein refers to any formulation suitable for oral administration, including but not limited to tablets, capsules, films, wafers, and suspensions.

“Gastrointestinal mucosa” or “GI mucosa” as used herein refers to any mucus-lined portion of the GI tract, including but not limited to the stomach, small intestines, and large intestines

“Magnetic material” as used herein refers to any material that induces a force or movement when introduced into a magnetic field.

“Magnetomucoadhesion” as generally used herein refers to the retention of a magnetic material containing dosage form at a site within the gastrointestinal tract via the application of an extracorporeal magnet and by the mucoadhesive forces generated between the GI mucosa and the dosage form that allows for adhesion both during and after the application of an extracorporeal magnet.

“Magnetomucoadhesive materials” as generally used herein refers to materials that exhibit mucoadhesive properties that also exhibit magnetic properties.

“Magnetic material containing dosage” as generally used herein refers to an orally administered formulation containing a magnetic material.

“Metallomucoadhesion” as generally used herein refers to the retention of a metal containing material at a site within the gastrointestinal tract, whether in vitro or in vivo.

“Mucoadhesive polymers” as generally used herein mean polymers that have an adherence to living mucosal tissue of at least about 110 N/m² of contact area (11 mN/cm²). A suitable measurement method is set forth in U.S. Pat. No. 6,235,313 to Mathiowitz et al.

“Site-directed” as generally used herein refers to tissue-specific or organ-specific delivery via the oral route.

### II. FORMULATIONS

In one preferred embodiment, the formulations contain microparticles or nanoparticles and a drug (or drugs) to be delivered and, optionally, an appropriate carrier. The microparticles can be formed of the drug to be delivered alone or in combination with excipients, or on, in, or blended with a polymer, preferably a mucoadhesive polymer. The formulation may be in the form of a liquid such as a dispersion or suspension of microparticles or nanoparticles, or may be in a solid dosage form, such as tablets, capsules, multiparticulate formulations, beads, granules, or particles. The formulation may contain an enteric or non-enteric coating. Preferably the formulation is an oral dosage formulation. Additionally, the formulation may contain metal compounds. The metal compounds will be in the form of a micron-sized or sub-micron sized particles or may be a macro-sized particle. The metal compounds may be coated on the surface of the dosage form or inside the dosage form. Optionally, the particles may be blended with a polymer and/or excipients.

Populations of nanoparticles in specific size ranges in the formulation are delivered to particular sites in the GI tract. This results in desirable bioavailability and biodistribution profiles. Further, by magnetically directing the dosage to a site of absorption and retaining the dosage in close proximity to the digestive epithelium, the desired biodistribution can be achieved per orally and non-invasively. Increasing the fraction of the dose localized at the absorptive epithelium and its residence time leads to increased uptake and overall bioavailability of the encapsulated therapeutic agent

In another embodiment, the formulations may be a conventional controlled release system coated with or formulated to contain magnetic materials allowing for the localization and retention of drug delivery systems within the GI mucosa. Such controlled release systems include, but are not limited to, gelatin capsules with enteric coatings, tablet formulations, and osmotic-pump-based delivery systems. Such conventional controlled release systems optionally contain nanoparticles of the active agent to be delivered.

A. Polymers

Polymers included in the nanoparticles or microparticles are typically biodegradable polymers. In the preferred embodiment, the polymers are mucoadhesive polymers.

Representative polymers which can be used include hydrophilic polymers, such as those containing carboxylic groups, including polyacrylic acid. Bioerodible polymers including polyanhydrides, poly(hydroxy acids) and polyesters, as well as blends and copolymers thereof also can be used. Representative bioerodible poly(hydroxy acids) and copolymers thereof which can be used include poly(lactic acid), poly(glycolic acid), poly(hydroxy-butyric acid), poly(hydroxyvaleric acid), poly(caprolactone), poly(lactide-co-caprolactone), and poly(lactide-co-glycolide). Polymers containing labile bonds, such as polyanhydrides and polyorthoesters, can be used optionally in a modified form with reduced hydrolytic reactivity. Positively charged hydrogels, such as chitosan, and thermoplastic polymers, such as polystyrene, also can be used.

Representative natural polymers which also can be used include proteins, such as zein, modified zein, casein, gelatin, gluten, serum albumin, or collagen, and polysaccharides such as dextrans, polyhyaluronic acid and alginic acid. Representative synthetic polymers include polyphosphazenes, polyamides, polycarbonates, polyacrylamides, polysiloxanes, polyurethanes and copolymers thereof. Celluloses also can be used. As defined herein the term “celluloses” includes naturally occurring and synthetic celluloses, such as alkyl celluloses, cellulose ethers, cellulose esters, hydroxyalkyl celluloses and nitrocelluloses. Exemplary celluloses include ethyl cellulose, methyl cellulose, carboxymethyl cellulose, hydroxymethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, cellulose triacetate and cellulose sulfate sodium salt.

Polymers of acrylic and methacrylic acids or esters and copolymers thereof can be used. Representative polymers which can be used include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other polymers which can be used include polyalkylenes such as polyethylene and polypropylene; polyarylalkylenes such as polystyrene; poly(alkylene glycols), such as poly(ethylene glycol); poly(alkylene oxides), such as poly(ethylene oxide); and poly(alkylene terephthalates), such as poly(ethylene terephthalate). Additionally, polyvinyl polymers can be used, which, as defined herein includes polyvinyl alcohols, polyvinyl ethers, polyvinyl esters and polyvinyl halides. Exemplary polyvinyl polymers include poly(vinyl acetate), polyvinyl phenol and polyvinylpyrrolidone.

Polymers which alter viscosity as a function of temperature or shear or other physical forces also may be used. Poly(oxyalkylene) polymers and copolymers such as poly(ethylene oxide)-poly(propylene oxide) (PEO-PPO) or poly(ethylene oxide)-poly(butylene oxide) (PEO-PBO) copolymers, and copolymers and blends of these polymers with polymers such as poly(alpha-hydroxy acids), including but not limited to lactic, glycolic and hydroxybutyric acids, polycaprolactones, and polyvalerolactones, can be synthesized or commercially obtained. For example, polyoxyalkylene copolymers, such as copolymers of polyoxyethylene and polyoxypropylene are described in U.S. Pat. Nos. 3,829,506; 3,535,307; 3,036,118; 2,979,578; 2,677,700; and 2,675,619.

Polyoxyalkylene copolymers are sold, for example, by BASF under the tradename Pluronics™. These materials are applied as viscous solutions at room temperature or lower which solidify at the higher body temperature. Other materials with this behavior are known in the art, and can be utilized as described herein. These include Klucel™ (hydroxypropyl cellulose), and purified konjac glucomaiman gum.

Polymer solutions that are liquid at an elevated temperature but solid or gelled at body temperature can also be utilized. A variety of thermoreversible polymers are known, including natural gel-forming materials such as agarose, agar, furcellaran, beta-carrageenan, beta-1,3-glucans such as curdlan, gelatin, or polyoxyalkylene containing compounds, as described above. Specific examples include thermosetting biodegradable polymers for in vivo use described in U.S. Pat. No. 4,938,763 to Dunn, et al.

These polymers can be obtained from sources such as Sigma Chemical Co., St. Louis, Mo.; Polysciences, Warrenton, Pa.; Aldrich, Milwaukee, Wis.; Fluka, Ronkonkoma, N.Y.; and BioRad, Richmond, Calif., or can be synthesized from monomers obtained from these or other suppliers using standard techniques.

Polyanhydrides are a preferred type of mucoadhesive polymer. Suitable polyanhydrides include polyadipic anhydride, polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios.

Blending or copolymerization sufficient to provide a certain amount of hydrophilic character in the polymer matrix can be useful to improve wettability of the materials. For example, about 5% to about 20% of monomers may be hydrophilic monomers, Hydrophilic polymers such as hydroxylpropylcellulose (HPC), hydroxpropylmethylcellulose (HPMC), carboxymethylcellulose (CMC) are commonly used for this purpose. Also suitable are hydrophobic polymers such as polyesters and polyimides. It is known to those skilled in the art that these polymers may be blended with polyanhydrides to achieve compositions with different drug release profiles and mechanical strengths. Preferably, the polymers are bioerodible, with preferred molecular weights ranging from 1000 to 15,000 kDa, and most preferably 2000 to 5000 Da.

Rate controlling polymers may be included in the polymer matrix or in the coating on the formulation. Examples of rate controlling polymers that may be used in the dosage form are hydroxypropylmethylcellulose (HPMC) with viscosities of either 5, 50, 100 or 4000 cps or blends of the different viscosities, ethylcellulose, methylmethacrylates, such as EUDRAGIT® RS100, EUDRAGIT® RL100, EUDRAGIT® NE 30D (supplied by Rohm America). Gastrosoluble polymers, such as EUDRAGIT® E100 or enteric polymers such as EUDRAGIT® L100-55D, L100 and S100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

B. Active Agents

One or more active agents may be formulated alone or with excipients or encapsulated on, in or incorporated into the microparticles or nanoparticles. Active agents include therapeutic, prophylactic, and diagnostic agents. Any suitable agent may be used. These include organic compounds, inorganic compounds, proteins, polysaccharides, nucleic acids or other materials that can be incorporated using standard techniques.

Active agents include synthetic and natural proteins (including enzymes, peptide-hormones, receptors, growth factors, antibodies, signaling molecules), and synthetic and natural nucleic acids (including RNA, DNA, anti-sense RNA, triplex DNA, inhibitory RNA (RNAi), and oligonucleotides), and biologically active portions thereof. Suitable active agents have a size greater than about 1,000 Da for small peptides and polypeptides, more typically at least about 5,000 Da and often 10,000 Da or more for proteins. Nucleic acids are more typically listed in terms of base pairs or bases (collectively “bp”). Nucleic acids with lengths above about 10 bp are typically used in the present method. More typically, useful lengths of nucleic acids for probing or therapeutic use will be in the range from about 20 bp (probes; inhibitory RNAs, etc.) to tens of thousands of bp for genes and vectors. The active agents may also be hydrophilic molecules, preferably having a low molecular weight.

Examples of useful proteins include hormones such as insulin and growth hormones including somatomedins. Examples of useful drugs include neurotransmitters such as L-DOPA, antihypertensives or saluretics such as Metolazone from Searle Pharmaceuticals, carbonic anhydrase inhibitors such as Acetazolamide from Lederle Pharmaceuticals, insulin like drugs such as glyburide, a blood glucose lowering drug of the sulfonylurea class, synthetic hormones such as Android F from Brown Pharmaceuticals and Testred® (methyltestosterone) from ICN Pharmaceuticals.

Under the Biopharmaceutical Classification System (BCS), drugs can belong to four classes: class I (high permeability, high solubility), class II (high permeability, low solubility), class III (low permeability, high solubility) or class IV (low permeability, low solubility). Suitable active agents also include poorly soluble compounds; such as drugs that are classified as class II or class IV compounds using the BCS. Examples of class II compounds include: acyclovir, nifedipine, danazol, ketoconazole, mefenamic acid, nisoldipine, nicardipine, felodipine, atovaquone, griseofulvin, troglitazone glibenclamide and carbamazepine. Examples of class IV compounds include: chlorothiazide, furosemide, tobramycin, cefuroxmine, and paclitaxel.

For imaging, radioactive materials such as Teclmetium99 (^{99m}Tc) or magnetic materials such as γ-Fe₂O₃ could be used. Examples of other materials include gases or gas emitting compounds, which are radioopaque.

The biologically active agents can be micronized to form small particles that retain a significant and therapeutically useful level of recoverable biologic activity. Preferably, the preparation retains at least 50% of it original biological activity, and more preferably the preparation retains 60-90% of its original biological activity, based on the weight of biologically active agent in the sample compared to an equal weight of the original biologically active agent. In the most preferred embodiment, the preparation retains greater than 90% of its original biological activity.

C. Excipients

The compositions may include a physiologically or pharmaceutically acceptable carrier, excipient, or stabilizer. The term “pharmaceutically acceptable” means a non-toxic material that does not interfere with the effectiveness of the biological activity of the active ingredients. The term “pharmaceutically-acceptable carrier” means one or more compatible solid or liquid fillers, dilutants or encapsulating substances which are suitable for administration to a human or other vertebrate animal. The term “carrier” refers to an organic or inorganic ingredient, natural or synthetic, with which the active ingredient is combined to facilitate the application.

The active compounds (or pharmaceutically acceptable salts thereof) may be administered in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (POLYPLASDONE® XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; diallyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The compounds may be complexed with other agents as part of their being pharmaceutically formulated. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

Delayed release and extended release compositions can be prepared. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT®(Röhm Pharma, Darmstadt, Germany), zein, shellac, and polysaccharides. Additionally, the coating material may contain conventional carriers such as plasticizers, pigments, colorants, glidants, stabilization agents, pore formers and surfactants.

Examples of rate controlling polymers that may be used in the dosage form are hydroxypropylmethylcellulose (HPMC) with viscosities of either 5, 50, 100 or 4000 cps or blends of the different viscosities, ethylcellulose, methylmethacrylates, such as EUDRAGIT®RS100, EUDRAGIT®RL100, EUDRAGIT®NE 30D (supplied by Rohm America). Gastrosoluble polymers, such as EUDRAGIT®E100 or enteric polymers such as EUDRAGIT®L100-55D, L100 and S100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers. Examples of suitable enteric coatings and the corresponding target region for release localized control are listed in Table 1.

TABLE 1
Enteric Coatings

| | Soluble | |
|---|---|---|
| Name | pH | Target release region |
| EUDRAGIT L 100-55 | >5.5 | Duodenum |
| EUDRAGIT L 30 D-55 | >5.5 | Duodenum |
| EUDRAGIT L 100 | >6.0 | Jujenum-Ileum |
| EUDRAGIT L 100/S 100 | >6.5 | Ileum |
| EUDRAGIT S 100 | >7.0 | Colon |
| EUDRAGIT FS 30 D | >7.0 | Colon |
| EUDRAGIT L 12,5 | >6.0 | Jujenum |
| EUDRAGIT S 12,5 | >7.0 | Colon |
| EUDRAGIT NE 30 D | swellable | Duodenum-Jejunum |
| EUDRAGIT NE 40 D | swellable | Ileum-Colon |
| EUDRAGIT RL 30 D | swellable | Stomach |
| EUDRAGIT RL PO | swellable | Stomach |
| EUDRAGIT RL 100 | swellable | Ileum |
| EUDRAGIT RS 30 D | swellable | Duodenum-Colon |
| EUDRAGIT RS PO | swellable | Duodenum-Colon |
| EUDRAGIT RS 100 | swellable | Jejunum-Colon |
| EUDRAGIT E 100 | <5.0 | Stomach |
| EUDRAGIT E PO | <5.0 | Stomach |

Swellable EUDRAGIT® is pH independent, time dependent.

D. Metallo- and Magneto-Mucoadhesive Materials for Localization

Metallomucoadhesive Materials for Localization

To increase mucoadhesion of the dosage formulation, elemental metals, including but not limited to, chromium, iron, titanium, aluminum, nickel, zinc, neodymium, magnesium, palladium, gold, silver, copper, vanadium, and their alloys can be incorporated into the formulation or coated on the surface of the formulation in an effective amount to increase mucoadhesion. If the metal compound is incorporated inside the formulation, it will typically be located beneath an enteric coating or in a degradable layer. The enteric coating will dissolve or the degradable layer will degrade under certain conditions and thereby expose the metal compounds to the mucosal surface where delivery is desired.

Magnetic Materials for Localization

Magnetic materials suitable for site-directed delivery can be incorporated in the coating of an oral dosage formulation or inside the oral dosage formulation and used for site-directed delivery. Suitable magnetic materials include, but are not limited to, ferromagnetica and superparamagnetic materials, such as iron containing compounds, martensitic stainless steels (e.g. 400 series), iron oxides (Fe₂O₃, Fe₃O₄), neodymium iron boron, alnico (AlNiCo), and samarium cobalt (SmCo₅). Moreover, individual magnetic materials have been shown to possess mucoadhesive properties indicating combined magnetic and mucoadhesive effects for achieving localized delivery. Mucoadhseive ferromagnetic and superparamagnetic compounds include but are not limited to iron-containing compounds such as martensitic stainless steels (e.g. 400-series), iron and iron oxides (Fe₂O₃, Fe₃O₄).

If the dosage formulation is a solid oral dosage form, such as a capsule, tablet or film the magnetic materials may be included in the coating of the capsule, tablet or film or inside the capsule, tablet or film.

In one embodiment, the magnetic material is in the form of micron-sized or sub-micron-sized particles. Such particles may be incorporated in micro or nano-particles, optionally the micro or nano-particles contain an active agent to be delivered. Suitable sizes for the magnetic material range from nanometers up to centimeters in cross-sectional diameter or width.

In another embodiment, the magnetic material is larger than 10 microns in length, width, and/or diameter, and may have any shape, such as a cylinder, rectangular box, cube, sphere, disc, or ring.

By controlling microsphere size and site of delivery (via enteric coatings and magnetic particles) the uptake and biodistribution profiles can be controlled. This has great implications for the fabrication of a targeted oral delivery system.

E. Conventional Controlled Release Systems

In one embodiment, the formulations may be a conventional controlled release system coated with or formulated to contain magnetic materials allowing for the localization and retention of drug delivery systems within the GI mucosa. Such controlled release systems include, but are not limited to, gelatin capsules, capsules, tablets, and osmotic-pump-based delivery systems, optionally with an enteric coating. Such conventional controlled release systems may also contain nanoparticles of the active agent to be delivered. For example, a magnetic material may be placed into a capsular dosage form (e.g. a gelatin capsule) along with a therapeutic, diagnostic or prophylactic agent to be delivered, optionally with one or more excipients.

### III. KITS

Dosage formulations containing metals, as described herein, may be provided in the form of a kit. Kits typically contain the dosage formulation to be administered along with an extracorporeal magnet. The extracorporeal magnet may be in any suitable carrier for placement on a surface of the body. Suitable carriers include flexible polymeric materials, woven materials, patches, preferably an adhesive patch, bracelets, key chains, etc. The magnet may be applied to the surface of the body alone, without a carrier. The magnet may have any shape, such as a cylinder, rectangular box, cube, sphere, disc, or ring.

Suitable magnetic materials for the extracorporeal magnet include but are not limited to iron, its oxides and salts, neodymium iron boron, aluminum-nickel-cobalt, and samarium cobalt magnets alone or as a composite material with any combination of non-magnetic metals, ceramics, or polymers. In particular ferromagnetic and superparamagnetic compounds provide for magnetic site-direction as a means of localization. Mucoadhesive ferromagnetic and superparamagnetic compounds include but are not limited to iron-containing compounds such as martensitic stainless steels (e.g. 400-series), iron oxides (Fe₂O₃, Fe₃O₄), neodymium iron boron ceramic (Nd₂Fe₁₄B) (NIB), as well as iron-free magnetic materials including alnico (AlNiCo) and samarium cobalt (SmCo₅) ceramics. Preferably the extracorporeal magnet is an NIB magnet due to its high magnetic field strength (BHₘₐₓ). NIB magnets are currently the strongest commercially available permanent magnets.

### IV. METHODS OF FORMING THE MICROPARTICLES AND NANOPARTICLES

Many different processes can be used to form the microparticles and nanoparticles. If the process does not produce particles having a homogenous size range, then the particles will be separated to produce a population of particles having the desired size range.

A. Solvent Evaporation

Methods for forming microspheres using solvent evaporation techniques are described in E. Mathiowitz et al., J. Scanning Microscopy, 4:329 (1990); L. R. Beck et al., Fertil. Steril., 31:545 (1979); L. R. Beck et al Am J Obstet Gynecol 135(3) (1979); S. Benita et al., J. Pharm. Sci., 73:1721 (1984); and U.S. Pat. No. 3,960,757 to Morishita et al. The polymer is dissolved in a volatile organic solvent, such as methylene chloride. A substance to be incorporated optionally is added to the solution, and the mixture is suspended in an aqueous solution that contains a surface active agent such as poly(vinyl alcohol). Substances which can be incorporated in the microspheres include pharmaceuticals, pesticides, nutrients, imaging agents, metal elements, metal compounds, metal alloys, and magnetic materials. The resulting emulsion is stirred until most of the organic solvent evaporated, leaving solid microspheres. Microspheres with different sizes (1-1000 microns) and morphologies can be obtained by this method. This method is useful for relatively stable polymers like polyesters and polystyrene. However, labile polymers, such as polyanhydrides, may degrade during the fabrication process due to the presence of water. For these polymers, some of the following methods performed in completely anhydrous organic solvents are more useful.

B. Hot Melt Microencapsulation

Microspheres can be formed from polymers such as polyesters and polyanhydrides using hot melt microencapsulation methods as described in Mathiowitz et al., Reactive Polymers, 6:275 (1987). In this method, the use of polymers with molecular weights between 3-75,000 daltons is preferred. In this method, the polymer first is melted and then mixed with the solid particles of a substance to be incorporated that have been sieved to less than 50 microns. Substances which can be incorporated in the microspheres include pharmaceuticals, pesticides, nutrients, imaging agents, and metal compounds. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microspheres are washed by decanting with petroleum ether to give a free-flowing powder. Microspheres with sizes between one to 1000 microns are obtained with this method.

C. Solvent Extraction

This technique is primarily designed for polyanhydrides and is described, for example, in WO 93/21906 to Brown University Research Foundation. In this method, the substance to be incorporated is dispersed or dissolved in a solution of the selected polymer in a volatile organic solvent, such as methylene chloride. This mixture is suspended by stirring in an organic oil, such as silicon oil, to form an emulsion. Microspheres that range between 1-300 microns can be obtained by this procedure. Substances which can be incorporated in the microspheres include pharmaceuticals, pesticides, nutrients, imaging agents, and metal compounds.

D. Spray-Drying

Methods for forming microspheres using spray drying techniques are described in U.S. Pat. No. 6,620,617, to Mathiowitz et al. In this method, the polymer is dissolved in an organic solvent such as methylene chloride or in water. A known amount of an agent to be incorporated is suspended (insoluble agent) or co-dissolved (soluble agent) in the polymer solution. The solution or the dispersion then is spray-dried. Microspheres ranging between 0.1-10 microns are obtained. This method is useful for preparing microspheres for imaging of the intestinal tract. Using the method, diagnostic imaging agents such as gases can be incorporated into the microspheres. Substances which can be incorporated in the microspheres include pharmaceuticals, pesticides, nutrients, imaging agents, and metal compounds.

E. Phase Inversion

Microspheres can be formed from polymers using a phase inversion method wherein a polymer is dissolved in a “good” solvent, fine particles of a substance to be incorporated, such as a drug, are mixed or dissolved in the polymer solution, and the mixture is poured into a strong non-solvent for the polymer, to spontaneously produce, under favorable conditions, polymeric microspheres, wherein the polymer is either coated with the particles or the particles are dispersed in the polymer. The method can be used to produce microparticles in a wide range of sizes, including, for example, about 100 nanometers to about 10 microns. Exemplary polymers which can be used include polyvinylphenol and polylactic acid. Substances which can be incorporated include, for example, imaging agents such as fluorescent dyes, or biologically active molecules such as proteins or nucleic acids. In the process, the polymer is dissolved in an organic solvent and then contacted with a non-solvent, which causes phase inversion of the dissolved polymer to form small spherical particles, with a narrow size distribution optionally incorporating a drug or other substance. Substances which can be incorporated in the microspheres include pharmaceuticals, pesticides, nutrients, imaging agents, and metal compounds.

Advantageously, an emulsion need not be formed prior to precipitation. The process can be used to form microspheres from thermoplastic polymers such as those listed in Table 2 below. Table 2 shows the results of phase inversion experiments including: correlation of polymer species, molecular weight, concentration, viscosity, solvent:non-solvent pairs and final product morphology. Viscosity units are centipoise and concentration units are (w/v) referring to initial polymer concentration.

TABLE 2
List of Thermoplastic polymers

| Polymer | MW | | Conc | Visc | Solvent | Non-Solvent | Product |
|---|---|---|---|---|---|---|---|
| Poly(adipic | 7 | kDa | 1% | | methylene | petroleum | 1-6 μm |
| anhydride) | | | | | chloride | ether | |
| Poly(adipic | 7 | kDa | 2% | | methylene | petroleum | 1-8 μm |
| anhydride) | | | | | chloride | ether | |
| Poly(adipic | 7 | kDa | 5% | | methylene | petroleum | 1-15 μm |
| anhydride) | | | | | chloride | ether | |
| Poly(adipic | 7 | kDa | 10% | | methylene | petroleum | 1-30 μm |
| anhydride) | | | | | chloride | ether | |
| polystyrene | 50 | kDa | 1% | | methylene | petroleum | 500 nm-2 μm |
| | | | | | chloride | ether | |
| polystyrene | 50 | kDa | 3% | | methylene | petroleum | 1-2 μm |
| | | | | | chloride | ether | |
| polystyrene | 50 | kDa | 5% | | methylene | petroleum | 1-4 μm |
| | | | | | chloride | ether | |
| polystyrene | 50 | kDa | 10% | | methylene | petroleum | 1-5 μm |
| | | | | | chloride | ether | |
| polystyrene | 50 | kDa | 15% | | methylene | petroleum | 1-10 μm & |
| | | | | | chloride | ether | aggregates |
| polystyrene | 50 | kDa | 20% | | methylene | petroleum | large aggregates |
| | | | | | chloride | ether | |
| polystyrene | 50 | kDa | 1% | | methylene | ethanol | <100 nm |
| | | | | | chloride | | |
| polystyrene | 50 | kDa | 5% | | methylene | ethanol | <100 nm |
| | | | | | chloride | | |
| polystyrene | 50 | kDa | 10% | | methylene | ethanol | 100 nm-3 μm |
| | | | | | chloride | | |
| polycapro- | 72 | kDa | 1% | 3.188 | methylene | petroleum | 1-3 μm |
| lactone | | | | | chloride | ether | |
| polycapro- | 72 | kDa | 5% | 7.634 | methylene | petroleum | large aggregates |
| lactone | | | | | chloride | ether | |
| polycapro- | 112 | kDa | 1% | 4.344 | methylene | petroleum | aggregates |
| lactone | | | | | chloride | ether | |
| polycapro- | 112 | kDa | 5% | | methylene | ethanol | large aggregates |
| lactone | | | | | chloride | | |
| polyvinyl- | 1.5-7 | kDa | 1% | | acetone | petroleum | 250 nm-1 μm |
| phenol | | | | | | ether | |
| polyvinyl- | 1.5-7 | kDa | 5% | | acetone | petroleum | 1-2 μm |
| phenol | | | | | | ether | |
| polyvinyl- | 1.5-7 | kDa | 10% | | acetone | petroleum | 1-5 μm |
| phenol | | | | | | ether | |
| polyvinyl- | 9-11 | kDa | 1% | | acetone | petroleum | 100 nm-2 μm |
| phenol | | | | | | ether | |
| polyvinyl- | 9-11 | kDa | 5% | | acetone | petroleum | 250 nm-2.5 μm |
| phenol | | | | | | ether | |
| polyvinyl- | 9-11 | kDa | 10% | | acetone | petroleum | 500 nm-10 μm |
| phenol | | | | | | ether | |
| polylactic acid | 2 | kDa | 1% | 0.876 | methylene | petroleum | 100 nm |
| | | | | | chloride | ether | |
| polylactic acid | 2 | kDa | 5% | 1.143 | methylene | petroleum | 500 nm-2 μm |
| | | | | | chloride | ether | |
| polylactic acid | 2 | kDa | 10% | 2.299 | methylene | petroleum | 1-10 μm |
| | | | | | chloride | ether | |
| polylactic acid | 24 | kDa | 1% | 1.765 | methylene | petroleum | 100 nm |
| | | | | | chloride | ether | |
| polylactic acid | 24 | kDa | 5% | 2.654 | methylene | petroleum | 500 nm-1 μm |
| | | | | | chloride | ether | |
| polylactic acid | 24 | kDa | 10% | 3.722 | methylene | petroleum | 10 μm & |
| | | | | | chloride | ether | aggregates |
| polylactic acid | 100 | kDa | 1% | 2.566 | methylene | petroleum | 100 nm |
| | | | | | chloride | ether | |
| polylactic acid | 100 | kDa | 5% | 4.433 | methylene | petroleum | 0.5-2 μm & |
| | | | | | chloride | ether | aggregates |
| polylactic acid | 100 | kDa | 10% | 8.256 | methylene | petroleum | film & |
| | | | | | chloride | ether | aggregates |
| ethylenevinyl | 55 | kDa | 1% | | methylene | petroleum | globular strands |
| acetate | | | | | chloride | ether | |
| ethylenevinyl | 55 | kDa | 5% | | methylene | petroleum | coalesced strands |
| acetate | | | | | chloride | ether | |
| ethylenevinyl | 55 | kDa | 10% | | methylene | petroleum | continuous sheet |
| acetate | | | | | chloride | ether | |
| Poly(acrylo- | >100 | kDa | 1% | 2.566 | acetone | petroleum | 1-20 μm |
| nitrile-co- | | | | | | ether | |
| vinyl chloride | | | | | | | |
| Poly(acrylo- | >100 | kDa | 5% | 15.903 | acetone | petroleum | 100 μm & |
| nitrile-co- | | | | | | ether | aggregates |
| vinyl chloride | | | | | | | |

F. Protein Microencapsulation

Protein microspheres can be formed by phase separation in a non-solvent followed by solvent removal as described in U.S. Pat. No. 5,271,961 to Mathiowitz et al. Proteins which can be used include prolamines such as zein. Additionally, mixtures of proteins or a mixture of proteins and a bioerodible material polymeric material such as a polylactide can be used. In one embodiment, a prolamine solution and a substance to be incorporated are contacted with a second liquid of limited miscibility with the proline solvent, and the mixture is agitated to form a dispersion. The prolamine solvent then is removed to produce stable prolamine microspheres without crosslinking or heat denaturation. Other prolamines which can be used include gliadin, hordein and kafirin. Substances which can be incorporated in the microspheres include pharmaceuticals, pesticides, nutrients, imaging agents, and metal compounds.

G. Low Temperature Casting of Microspheres

Methods for very low temperature casting of controlled release microspheres are described in U.S. Pat. No. 5,019,400 to Gombotz et al. In the method, a polymer is dissolved in a solvent together with a dissolved or dispersed substance to be incorporated, and the mixture is atomized into a vessel containing a liquid non-solvent at a temperature below the freezing point of the polymer-substance solution, which freezes the polymer droplets. As the droplets and non-solvent for the polymer are warmed, the solvent in the droplets thaws and is extracted into the non-solvent, resulting in the hardening of the microspheres. Substances which can be incorporated in the microspheres include pharmaceuticals, pesticides, nutrients, imaging agents, and metal compounds.

H. Double Walled Microcapsules

Multiwall polymer microspheres may be prepared by dissolving two hydrophilic polymers in an aqueous solution. A substance to be incorporated is dispersed or dissolved in the polymer solution, and the mixture is suspended in a continuous phase. The solvent then is slowly evaporated, creating microspheres with an inner core formed by one polymer and an outer layer of the second polymer. The continuous phase can be either an organic oil, a volatile organic solvent, or an aqueous solution containing a third polymer that is not soluble with the first mixture of polymers and which will cause phase separation of the first two polymers as the mixture is stirred. Substances which can be incorporated in the microspheres include pharmaceuticals, pesticides, nutrients, imaging agents, and metal compounds.

Multilayer polymeric drug, protein, or cell delivery systems can be prepared from two or more hydrophilic polymers using the method. Any two or more different biodegradable, or non-degradable, water soluble polymers which are not soluble in each other at a particular concentration as dictated by their phase diagrams may be used. The multilayer microcapsules have uniformly dimensioned layers of polymer and can incorporate a range of substances in addition to the metal compound including biologically active agents such as drugs or cells, or diagnostic agents such as dyes.

Microspheres containing a polymeric core made of a first polymer and a uniform coating of a second polymer, and a substance incorporated into at least one of the polymers, can be made as described in U.S. Pat. No. 4,861,627.

I. Hydrogel Microspheres

Microspheres made of gel-type polymers, such as alginate, are produced through traditional ionic gelation techniques. The polymer first is dissolved in an aqueous solution, mixed with a substance to be incorporated, and then extruded through a microdroplet forming device, which in some instances employs a flow of nitrogen gas to break off the droplet. A slowly stirred ionic hardening bath is positioned below the extruding device to catch the forming microdroplets. The microspheres are left to incubate in the bath for twenty to thirty minutes in order to allow sufficient time for gelation to occur. Microsphere particle size is controlled by using various size extruders or varying either the nitrogen gas or polymer solution flow rates. Substances which can be incorporated in the microspheres include pharmaceuticals, pesticides, nutrients, imaging agents, and metal compounds.

Chitosan microspheres can be prepared by dissolving the polymer in acidic solution and crosslinking it with tripolyphosphate. Carboxymethyl cellulose (CMC) microspheres can be prepared by dissolving the polymer in acid solution and precipitating the microsphere with lead ions. Alginate/polyethylene imide (PEI) can be prepared in order to reduce the amount of carboxylic groups on the alginate microcapsule. The advantage of these systems is the ability to further modify their surface properties by the use of different chemistries. In the case of negatively charged polymers (e.g., alginate, CMC), positively charged ligands (e.g., polylysine, polyethyleneimine) of different molecular weights can be ionically attached.

Micronized oligomer particles can be mixed with the hydrogel solution before gelation or else the hydrogel microspheres may be lyophilized and coated with the oligomer solution by dipping or spraying.

### V. METHODS OF MAKING TABLETS AND OTHER SOLID ORAL DOSAGE FORMULATIONS

Many different processes can be used to form the solid oral dosage formulations. Some suitable processes are described below.

A. Film Manufacturing Procedure

Inactive ingredients are weighed out and placed in a beaker. A quantity of water is added (sufficient to achieve desired viscosity) and ingredients are mixed on a stir plate for at least one hour. Active ingredients are finally added to mixture just before pouring/pipetting to sheet/mold for drying. Films may be made as a large sheet and cut to desired size depending on the dosing, or individually dosed into molds at the desired dosage. In another film dosage form, the active ingredient may be in a separate layer in order to protect it from degradation over time. In a third form, a third layer could be inserted between the active and other ingredients to further protect the active from other potentially degrading ingredients located in the other layer during storage.

The film can be designed to dissolve rapidly (<30 seconds) or slowly (up to 15 minutes) in order to achieve the desired absorption profile and subsequent effect by altering the composition of inactive ingredients.

B. Tablets

Tablets are made using a traditional compression machine with flat punches. Dry active ingredients, optionally including a metallomucoadhesive material and/or magnetic material, are combined with a binding agent and added to the die. The depth of the tablet is determined by the quantity of ingredients. Compression should be kept to a minimum (sufficient to hold the ingredients together during dose administration, yet soft enough to allow water penetration into the tablet for easy dissolution in the mouth). The tablets may be composed of a single homogeneous powder or a bi-layer composed of two sets of ingredients.

C. Wafers

A compression machine may also be used to make wafers using a larger, flatter punch, or alternatively, the mixed dry materials could be flattened/compressed between rollers to form the powder into a sheet that may be cut to an appropriate size (to be inserted under the tongue). Dosing of the wafers could be determined by either the altering the concentration of the active in the powder and keeping the wafer size uniform, or simply keeping the concentration the powder uniform and increasing the surface area of the wafer to achieve higher doses.

Wafers can also be made by putting the dry powders into aqueous solution, pipetting the appropriate amount of solution into molds, flash freezing and lyophilizing the material. This forms a very light wafer that dissolves very rapidly and requires little fill and binding material.

### VI. METHODS OF SITE-DIRECTED DELIVERY

Oral dosage formulations are administered to a patient. In one embodiment, following or at the time of administration, an extracorporeal magnet is placed on the outside surface of the body in an area that closely apposes the location in the gastrointestinal tract to which delivery of the formulation is desired. In one embodiment, the extracorporeal magnet is placed at this site for a suitable period of time to induce mucoadhesion between the formulation and the site of delivery and then the magnet is removed. The formulation containing magnetic material can be imbedded in the mucosa of intestinal tissue and will maintain the adherence to the mucosa at the tissue site, even after the magnetic field has been removed. Suitable time periods for placing the extracorporeal magnet on the surface of the body range to attach the formulation to the GI mucosa range from 1 to 8 hours, preferably from 1 to 3 hours, more preferably from 2 to 3 hours

In another embodiment, the extracorporeal magnet is placed at this site for a suitable period of time to localize the formulation at the desired site. During this time period an active agent may be released from the formulation at the site. Suitable time periods for placing the extracorporeal magnet on the surface of the body range to release an active agent from the formulation range from 5 minutes to 24 hours.

In a preferred embodiment, the formulation contains microparticles and nanoparticles containing the therapeutic, prophylactic or diagnostic agent to be delivered, in the form of a homogenous population wherein preferably 75%, more preferably 80%, most preferably about 90% or more, have a mean diameter of about 0.5 micron (majority range between 400 and 500 nm). This size range is particularly preferred for delivery to the portal circulation and liver via the jejunum. Preferably, the formulation is site-directed to the jejunum for release of the therapeutic, prophylactic or diagnostic agent.

To target delivery to the lungs, the formulation preferably contains microparticles and nanoparticles containing the therapeutic, prophylactic or diagnostic agent to be delivered, in the form of a homogenous population wherein preferably 75%, more preferably 80%, most preferably about 90% or more, have a mean diameter of about one micron. In a preferred embodiment, the formulation is site-directed to the jejunum for release of the therapeutic, prophylactic or diagnostic agent.

In another preferred embodiment, the formulation contains microparticles and nanoparticles containing the therapeutic, prophylactic or diagnostic agent to be delivered, in the form of a homogenous population wherein preferably 75%, more preferably 80%, most preferably about 90% or more, have a mean diameter of about one micron (majority range between 0.5 and 2 microns). This size range is particularly preferred for delivery to the portal circulation and liver via the ileum. Preferably, the formulation is site-directed to the ileum for release of the therapeutic, prophylactic or diagnostic agent.

In another embodiment, the formulation is in the form of a conventional controlled release system oral dosage formulation, such as a tablet or capsule, containing a magnetic material and the formulations is site-directed to the desired location in the gastrointestinal tract for release of a therapeutic, prophylactic or diagnostic agent. After per oral administration, an extracorporeal magnet is applied to direct delivery to the desired site in the GI tract.

In one embodiment, magnetically triggered orifice formation may occur to release the drug from the controlled release system. In this embodiment, the extracorporeal magnet can be used to pull the magnetic material through the wall of the capsular dosage form creating a diffusion orifice for the therapeutic agent and its excipients. An orifice is created when the dosage form is sufficiently anchored to the GI mucosa due to mucoadhesive forces (e.g. by a mucoadhesive material) to withstand the force of magnetic attraction without generating acceleration. In this embodiment, the tensile strength of adhesion exceeds the attractive force at yield. Additionally, the stress generated by the magnetic material upon the interior of the dosage form must exceed the yield strength of the capsule wall. Under these conditions, magnetically triggered orifice formation may occur.

In place of metal compounds, the formulations may contain a mucoadhesive polymer that adhere to the desired site. Preferably, such formulations contain an enteric coating, which dissolves at the appropriate pH, exposing the mucoadhesive polymer.

The present invention will be further understood by reference to the following non-limiting examples.

### EXAMPLES

### Example 1

### Quantitative Biodistribution of Polystyrene Microspheres

Method

The following method was used to quantitatively analyze microsphere uptake and biodistribution to specific rat tissues.

Male Sprague-Dawley rats, weighing 175-200 g, were used throughout the study. Rats were fed standard rat feed and water ad libidum from time of arrival to time of study. Study animals were first anesthetized with isoflurane and maintained under anesthesia peri-operatively. A 6-7 cm midline abdominal incision was made to expose intestines. Small intestine (SI) regions were identified using the ligament of Trietz (proximal jejunum) and increased Peyer's patch content (distal jejunum) as markers. A 6 cm section of the desired intestinal region was selected and gently cleared of its continents. The section was then ligated with 0-0 silk sutures by threading the suture through the intestinal mesentery (away from blood vessels) and then tying off both ends of the section taking care not to occlude blood flow.

In Rats 1, 2, 3, and 4, a 1 mL suspension of polystyrene (“PS”) microspheres was injected with a 23 gauge needle into the isolated section. In Rat 5, 1 mL of saline (control) was injected with a 23 gauge needle into the jejunum. Upon removal of the needle, a cauterizer was used to prevent leakage of the microsphere suspension. While maintaining anesthesia, the rats were kept alive for five hours to allow uptake of microspheres. The lesion was stapled during this period to avoid excessive loss of body heat and moisture.

Following the five hour period, the lesion was re-opened and extended into the thoracic cavity to expose the entire tissue cavity. A 1 mL hepatic blood sample was obtained from the hepatic portal vein followed by a 1 mL central blood sample obtained from the right ventricle. After obtaining the blood samples, all tissues were harvested. The order of removal was as follows: (1) lungs, (2) heart, (3) spleen, (4) kidneys, (5) liver, (6) intestinal section, and (7) brain. The intestinal section was thoroughly rinsed with saline and kept for processing with tissues (intestinal content).

All tissues were then individually homogenized with an ultrasonic homogenizing tip until a paste was formed. Each sample, in paste form, was lyophilized for 72 hours yielding a powdered form of the sample. 5 mL of chloroform was then added to the powdered samples and polystyrene was extracted on an end-over-end mixer for 96 hours. The extract was then positive-pressure filtered and lyophilized for 24 hours yielding a powder form of all extracts.

Chloroform (1 mL) was added to the extract and mixed for 1 hour. These samples were then run over a GPC column. The quantity of microspheres in each tissue was determined based on a standard curve. The area under the characteristic polystyrene peak is linearly related to the concentration of microspheres in each tissue sample.

In Rat 6 the microspheres were given via an oral gavage. The non-fasted animal was administered the microsphere suspension orally. Over a five hour period, urine and feces were collected. Following the period samples were harvested under isoflurane anesthesia, as described above. Each intestinal section and its contents were harvested as separate samples in addition to the samples taken in the isolated loop protocol. The processing of these samples was performed with the same method as described above.

Results

Results of this study are presented in Table 3 below.

TABLE 3
Treatment Conditions and Biodistribution Results

| Animal | | | | | |
|---|---|---|---|---|---|
| Rat 1* | Rat 2* | Rat 3 | Rat 4 | Rat 5 | Rat 6 |

Demographics

| | | | | | | |
|---|---|---|---|---|---|---|
| Rat weight/g | 192 | 187 | 201 | 195 | 195 | — |
| Spheres/mg | 25.8 | 25.8 | 27.1 | 25.7 | control | 26.4 |
| Sphere size/nm | 500 | 500 | 1000 | 2000 | control | 1000 |
| Region of the | Ileum | Jejunum | Jejunum | Jejunum | Jejunum | Gavage |
| Small Intestine | | | | | | |
| Incubation | 5 | 5 | 5 | 5 | 5 | 5 |
| time/h | | | | | | |

Biodistribution/mg

| | | | | | | |
|---|---|---|---|---|---|---|
| Brain | 0 | 0 | 0 | 0 | 0 | 0 |
| Central blood | 0.6 | 0 | 3.4 | 0 | 0 | 0.09 |
| Heart | 3.3 | 0 | 0.4 | 0 | 0 | 0 |
| Isolated loop* | 20.7 | 20.7 | 2.3 | 10.7 | 0 | — |
| Kidneys | 0 | 0 | 0.3 | 0 | 0 | 0 |
| Liver | 0 | 2.7 | 0.5 | 0 | 0 | 1.69 |
| Loop contents | * | * | 10.2 | 8.97 | 0 | — |
| Lungs | 0 | 0 | 7.2 | 0 | 0 | 0 |
| Portal blood | 0 | 1.5 | 0.3 | 0.03 | 0 | 1.98 |
| Spleen | 2.9 | 0 | 1.2 | 0 | 0 | 0.05 |
| Stomach | — | — | — | — | — | 0 |
| Stomach | — | — | — | — | — | 0 |
| contents | | | | | | |
| Duodenum | — | — | — | — | — | 0 |
| Duodenum | — | — | — | — | — | 0 |
| contents | | | | | | |
| Jejunum | — | — | — | — | — | 0.03 |
| Jejunum | — | — | — | — | — | 5.3 |
| contents | | | | | | |
| Ileum | — | — | — | — | — | 0 |
| Ileum contents | — | — | — | — | — | 1.1 |
| Cecum | — | — | — | — | — | 0.43 |
| Cecum | — | — | — | — | — | 2.92 |
| contents | | | | | | |
| Colon | — | — | — | — | — | 0 |
| Colon contents | — | — | — | — | — | 12.69 |
| Feces | — | — | — | — | — | 0.49 |
| Detection Mass | 106.6 | 96.5 | 95.2 | 76.66 | N/A | 101.42 |
| Balance/% | | | | | | |
| Percent | 24.7 | 16.9 | 51.6 | 0.15 | — | 14.24 |
| Uptake/% | | | | | | |

*Isolated loop and loop contents were processed together in the indicated studies

As shown in Table 3, the role of microsphere size and location of uptake (and, hence, location of delivery) have a direct effect on the amount of microsphere uptake and the biodistribution of the microsphere following uptake.

The effect of microsphere size is illustrated in comparing rats 2, 3 and 4 of the table (corresponding to 0.5, 1 and 2 μm microspheres). The amount of uptake was greatest with 1 μm microspheres (51.6%), less with 0.5 μm microspheres (16.9%) and nearly no uptake with the 2 μm microspheres (<1%). The biodistribution of the microspheres also varied with the microsphere size. 0.5 μm microspheres isolated to the portal blood and liver, suggesting retention by the liver via a first pass mechanism. However, the larger 1 μm microspheres distributed throughout most tissues, concentrating mostly in the lung. In addition, a substantial fraction of the microspheres were still circulating in the central blood compartment after the 5 hour time period. Despite the 1 μm microspheres having a three-fold higher uptake, the difference in distribution cannot be interpreted as a result of liver saturation due to the fact that fewer 1 μm microspheres were present in the liver than what was found in the 0.5 μm study. Therefore, one can conclude that the difference in biodistribution profiles is a direct effect of the microsphere size; the only difference setting the two formulations apart.

In addition to the size parameter, the location of microsphere delivery directly affects the biodistribution profile. This can be seen best by comparing Rat 1 and Rat 2. In both studies, 0.5 μm microspheres were delivered to either the jejunum or ileum. The jejunum delivery resulted in isolated delivery to the portal blood and liver. However, the ileum delivery seemed to bypass the liver and resulted in microsphere presence in the central blood compartment, heart and spleen. The amount of uptake was slightly higher in the ileum, 24.7% versus 16.9%, but, again, this cannot account for the difference in biodistribution. Therefore, the location of microsphere delivery is a critical determinant of the biodistribution profile. It is possible that this difference is a result of the increased amount of Peyer's patches in the ileum (i.e. the microspheres are taken up via a different mechanism than in the jejunum). However, the mechanism of uptake has not definitively been proven for either region.

Finally, the biodistribution profile is determined by the location of delivery. An oral gavage study (Rat 6) of 1 μm microspheres had a much different biodistribution profile than the same microspheres delivered locally to the jejunum (Rat 3). The majority of the microspheres from the fed study were found in the liver and portal blood. In addition, the amount of uptake was much less in the oral fed model (14.24% versus 51.6%).

In summary, the size and location of microsphere uptake/delivery are important determinants of the final biodistribution of oral microsphere systems.

### Example 2

### Reproduction of Example 1 with Larger Sample Size and Statistical Analysis

The procedure used in Example 1 was repeated with a larger sample size to test the reproducibility of the data and its statistical significance. Four rats were tested for each set of conditions tested (n=4). Additionally, two controls were performed to further test the process. The first control was tissue harvested, following an isolated loop of only saline, and processed. No detection of PS was evident for any of these tissue samples. The second control was again tissue harvested following an isolated loop of saline with known amount of PS that was injected into the tissues prior to processing. In each control sample, we detected 100% of the dose.

Analysis of the uptake data is presented below and summarized in several tables each related to different regions and microsphere sizes. In each table, mean values with standard error mean (n=4 for every group) are displayed.

An “*” indicates p<0.05 and a “+” indicates p<0.01. Percent of dose refers to the amount (mg) of PS detected in a given tissue, divided by the total amount (mg) of PS administered to the animal. Percent of uptake refers to the amount (mg) of PS detected in a given tissue, divided by the total amount (mg) of PS uptake where total PS uptake is defined as the total amount (mg) of PS detected in all tissue samples, excluding any intestinal sections and their contents, divided by the total amount (mg) of PS administered to the animal. A mass balance was determined as the total amount (mg) of PS detected in all samples, divided by the total amount (mg) of PS administered to the animal.

Table 4 compares uptake of 4 sizes of microspheres delivered to the jejunum.

TABLE 4
Effect of particle size on jejunum uptake

| | 0.5 μm | | 1 μm | | 2 μm | | 5 μm |
|---|---|---|---|---|---|---|---|
| | % of | % of | % of | % of | % of | % of | % of |
| Tissue | Dose | Uptake | Dose | Uptake | Dose | Uptake | Dose |
| Brain | 0.02 ± 0.02 | 0.04 ± 0.04 | 0.00 ± 0.00 | 0.00 ± 0.00 | 0.00 ± 0.00 | 0.00 ± 0.00 | 0.00 ± 0.00 |
| Central | 0.07 ± 0.05 | 0.12 ± 0.08 | 3.51 ± 3.02 | 7.94 ± 5.92 | 0.09 ± 0.09 | 0.46 ± 0.46 | 0.17 ± 0.09 |
| Blood | | | | | | | |
| Heart | 0.14 ± 0.05 | 0.29 ± 0.10 | 0.87 ± 0.20 | 3.78 ± 0.80 | 0.87 ± 0.54 | 3.74 ± 2.26 | 0.28 ± 0.26 |
| Kidneys | 3.62 ± 2.41 | 9.77 ± 7.35 | 2.97 ± 1.61 | 11.86 ± 6.41 | 2.82 ± 1.03 | 12.86 ± 4.64 | 0.19 ± 0.19 |
| Liver | 36 ± 9.88* | 78.08 ± 8.29* | 9.01 ± 3.23 | 45.20 ± 13.88 | 11.97 ± 4.06 | 54.70 ± 18.58 | 15.46 ± 5.59 |
| Lungs | 0.48 ± 0.32 | 0.95 ± 0.59 | 8.49 ± 6.03 | 25.77 ± 10.67* | 0.31 ± 0.25 | 1.54 ± 1.26 | 0.81 ± 0.60 |
| Portal | 2.26 ± 1.17 | 8.90 ± 6.50 | 0.47 ± 0.28 | 2.32 ± 1.66 | 0.24 ± 0.20 | 25.84 ± 24.73 | 0.97 ± 0.60 |
| blood | | | | | | | |
| Spleen | 0.63 ± 0.25 | 1.16 ± 0.40 | 1.24 ± 1.06 | 3.13 ± 2.00 | 0.20 ± 0.16 | 0.86 ± 0.67 | 0.43 ± 0.22 |

| | | | | |
|---|---|---|---|---|
| Mass | 101.67 ± 6.16 | 92.47 ± 1.22 | 97.05 ± 7.86 | 92.39 ± 6.06 |
| Balance | | | | |
| Total | 45.78 ± 8.64* | 28.90 ± 8.45* | 16.03 ± 5.46 | 19.40 ± 4.72 |
| uptake | | | | |

As shown by the data in Table 4, the amount of total uptake is inversely proportional to microsphere size, ranging from 45% for 0.5-micron microspheres to 19% for 5 micron microspheres. 0.5, 2 and 5 μm microspheres distributed primarily in the liver with very high percentage of the total dose; the highest amount was delivered to the liver with 0.5 um microspheres. In addition, 1 μm microspheres distributed to both the liver and lung. This difference in biodistribution is a result of particle size and is more evident when the data is viewed as the percent of uptake. Table 5 compares the effect of particle size on distribution of PS microspheres delivered to the ileum.

TABLE 5
Size comparison in ileum isolated loop

| | 0.5 μm | | 1 μm | | 2 μm | | 5 μm |
|---|---|---|---|---|---|---|---|
| | % of | % of | % of | % of | % of | % of | % of |
| Tissue | Dose | Uptake | Dose | Uptake | Dose | Uptake | Dose |
| Brain | 0.00 ± 0.00 | 0.00 ± 0.00 | 0.00 ± 0.00 | 0.00 ± 0.00 | 2.09 ± 2.09 | 22.27 ± 22.27 | 0.00 ± 0.00 |
| Central | 0.86 ± 0.50 | 3.57 ± 2.12 | 0.04 ± 0.03 | 0.15 ± 0.09 | 1.17 ± 0.71 | 22.31 ± 11.89 | 0.11 ± 0.08 |
| Blood | | | | | | | |
| Heart | 3.73 ± 3.03 | 14.69 ± 12.37 | 0.41 ± 0.23 | 1.14 ± 0.63 | 0.12 ± 0.08 | 3.20 ± 2.72 | 0.30 ± 0.18 |
| Kidneys | 0.35 ± 0.35 | 2.18 ± 2.18 | 1.01 ± 1.01 | 1.77 ± 1.77 | 0.11 ± 0.10 | 1.80 ± 1.51 | 3.98 ± 0.96 |
| Liver | 26.6 ± 14.20 | 54.13 ± 25.42 | 27.78 ± 10.35* | 78.25 ± 11.27 | 2.12 ± 1.23 | 39.01 ± 18.75 | 7.80 ± 4.02 |
| Lungs | 0.13 ± 0.08 | 0.46 ± 0.28 | 1.55 ± 0.99 | 12.89 ± 8.20 | 0.51 ± 0.34 | 7.13 ± 5.24 | 0.21 ± 0.21 |
| Portal | 1.63 ± 1.63 | 9.99 ± 9.99 | 0.28 ± 0.28 | 0.49 ± 0.49 | 0.12 ± 0.12 | 1.82 ± 1.78 | 0.12 ± 0.07 |
| blood | | | | | | | |
| Spleen | 3.72 ± 2.62 | 16.63 ± 10.72 | 0.47 ± 0.24 | 5.31 ± 4.66 | 0.16 ± 0.16 | 2.44 ± 2.44 | 0.00 ± 0.00 |

| | | | | |
|---|---|---|---|---|
| Mass | 106.02 ± 3.99 | 100.82 ± 3.60 | 105.33 ± 1.13 | 93.79 ± 3.02 |
| Balance | | | | |
| Total | 34.90 ± 9.29* | 32.18 ± 11.49* | 6.04 ± 1.19 | 13.39 ± 5.27 |
| uptake | | | | |

As shown by the data in Table 5, 0.5 and 1 μm microspheres have significant uptake, but do not differ from each other. 2 and 5 μm microspheres have relatively no uptake. Unlike the inverse relationship between size and uptake seen in the jejunum, the ileum seems to have a size threshold similar to a filtration system. The majority of microspheres isolate in the liver for 0.5 and 1 μm microspheres. While there is size dependence for uptake of microspheres, particles of varying sizes will have similar biodistribution after uptake. This is evident from the percent of uptake data in Table 4. A significant amount of 5 μm microspheres distribute to the kidneys and is a result of size as it remains significant when the data is corrected for total uptake. However, the very low uptake makes the total amount in the kidney negligible.

The next sets of studies compare the amount of uptake of the smallest size microspheres tested when delivered orally. Table 6 summarizes the data.

TABLE 6
Size comparison in oral gavage

| | 0.5 μm | | 1 μm |
|---|---|---|---|
| Tissue | % of Dose | % of Uptake | % of Dose |
| Brain | 2.56 ± 2.56 | 12.06 ± 12.06 | 0.00 ± 0.00 |
| Central blood | 0.07 ± 0.07 | 0.90 ± 0.90 | 0.09 ± 0.09 |
| Heart | 0.20 ± 0.12 | 1.11 ± 0.59 | 0.27 ± 0.16 |
| Kidneys | 1.80 ± 0.73 | 9.55 ± 5.24 | 1.12 ± 0.65 |
| Liver | 22.89 ± 9.81 | 71.69 ± 12.39 | 17.37 ± 7.33 |
| Lungs | 0.77 ± 0.56 | 3.36 ± 1.84 | 0.06 ± 0.06 |
| Portal blood | 0.00 ± 0.00 | 0.00 ± 0.00 | 2.65 ± 1.71 |
| Spleen | 0.01 ± 0.01 | 0.02 ± 0.02 | 0.05 ± 0.05 |
| Mass Balance | 85.04 ± 12.07 | 97.02 ± 2.31 | 97.02 ± 2.31 |
| Total uptake | 34.04 ± 9.53 | 22.45 ± 7.36 | 22.45 ± 7.36 |

As shown by the data in Table 6, the amount of uptake is inversely proportional to microsphere size. Regardless of microsphere size, the majority of microspheres distribute to the liver. This result may be useful in the design of insulin delivery systems.

Table 7 summarizes the distribution of the 0.5 μm microspheres in the three forms of administration, i.e. local delivery to the jejunum via injection, local delivery to the ileum via injection, and oral gavage.

TABLE 7
Location comparison for 0.5 μm microspheres

| | Jejunum | | Ileum | | Oral Gavage | |
|---|---|---|---|---|---|---|
| | % of | % of | % of | % of | | % of |
| Tissue | Dose | Uptake | Dose | Uptake | % of Dose | Uptake |
| Brain | 0.02 ± 0.02 | 0.04 ± 0.04 | 0.00 ± 0.00 | 0.00 ± 0.00 | 2.56 ± 2.56 | 12.06 ± 12.06 |
| Central blood | 0.07 ± 0.05 | 0.12 ± 0.08 | 0.86 ± 0.50 | 3.57 ± 2.12 | 0.07 ± 0.07 | 0.90 ± 0.90 |
| Heart | 0.14 ± 0.05 | 0.29 ± 0.10 | 3.73 ± 3.03 | 14.69 ± 12.37 | 0.20 ± 0.12 | 1.11 ± 0.59 |
| Kidneys | 3.62 ± 2.41 | 9.77 ± 7.35 | 0.35 ± 0.35 | 2.18 ± 2.18 | 1.80 ± 0.73 | 9.55 ± 5.24 |
| Liver | 36.73 ± 9.88 | 78.08 ± 8.29 | 26.6 ± 14.20 | 54.13 ± 25.42 | 22.89 ± 9.81 | 71.69 ± 12.39 |
| Lungs | 0.48 ± 0.32 | 0.95 ± 0.59 | 0.13 ± 0.08 | 0.46 ± 0.28 | 0.77 ± 0.56 | 3.36 ± 1.84 |
| Portal blood | 2.26 ± 1.17 | 8.90 ± 6.50 | 1.63 ± 1.63 | 9.99 ± 9.99 | 0.00 ± 0.00 | 0.00 ± 0.00 |
| Spleen | 0.63 ± 0.25 | 1.16 ± 0.40 | 3.72 ± 2.62 | 16.63 ± 10.72 | 0.01 ± 0.01 | 0.02 ± 0.02 |

| | | | |
|---|---|---|---|
| Mass Balance | 101.67 ± 6.16 | 106.02 ± 3.99 | 85.04 ± 12.07 |
| Total uptake | 45.78 ± 8.64 | 34.90 ± 9.29 | 34.04 ± 9.53 |

As shown by the data presented in Table 7, uptake was highest following local delivery to the jejunum with no significant difference in uptake between local delivery to the ileum and oral gavage. In all locations, 0.5 μm microspheres distributed primarily to the liver. There was also some distribution to the heart when delivered locally to the ileum, but this was not statistically significant.

Table 8 summarizes the distribution of the 1 um microspheres in the three methods of administration, i.e. local delivery to the jejunum via injection, local delivery to the ileum via injection, and oral gavage.

TABLE 8
Location comparison for 1 μm microspheres

| | Jejunum | | Ileum | | Oral Gavage | |
|---|---|---|---|---|---|---|
| | % of | % of | % of | % of | % of | % of |
| Tissue | Dose | Uptake | Dose | Uptake | Dose | Uptake |
| Brain | 0.00 ± 0.00 | 0.00 ± 0.00 | 0.00 ± 0.00 | 0.00 ± 0.00 | 0.00 ± 0.00 | 0.00 ± 0.00 |
| Central blood | 3.51 ± 3.02 | 7.94 ± 5.92 | 0.04 ± 0.03 | 0.15 ± 0.09 | 0.09 ± 0.09 | 0.61 ± 0.61 |
| Heart | 0.87 ± .20 | 3.78 ± 0.80 | 0.41 ± 0.23 | 1.14 ± 0.63 | 0.27 ± 0.16 | 1.01 ± 0.61 |
| Kidneys | 2.97 ± 1.61 | 11.86 ± 6.41 | 1.01 ± 1.01 | 1.77 ± 1.77 | 1.12 ± 0.65 | 4.79 ± 3.29 |
| Liver | 9.01 ± 3.23 | 45.20 ± 13.88 | 27.78 ± 10.35 | 78.25 ± 11.27 | 17.37 ± 7.33 | 74.56 ± 10.45 |
| Lungs | 8.49 ± 6.03 | 25.77 ± 10.67 | 1.55 ± 0.99 | 12.89 ± 8.20 | 0.06 ± 0.06 | 0.15 ± 0.15 |
| Portal blood | 0.47 ± 0.28 | 2.32 ± 1.66 | 0.28 ± 0.28 | 0.49 ± 0.49 | 2.65 ± 1.71 | 18.56 ± 11.88 |
| Spleen | 1.24 ± 1.06 | 3.13 ± 2.00 | 0.47 ± 0.24 | 5.31 ± 4.66 | 0.05 ± 0.05 | 0.32 ± 0.32 |

| | | | |
|---|---|---|---|
| Mass Balance | 92.47 ± 1.22 | 100.82 ± 3.60 | 97.02 ± 2.31 |
| Total uptake | 28.90 ± 8.45 | 32.18 ± 11.49 | 22.45 ± 7.36 |

As shown by the data in Table 8, there were no statistically significant differences in uptake of 1 μm microspheres in all administrations. In each form of administration, 1 μm microspheres distributed primarily to the liver. In local delivery to the jejunum there was also distribution to the lungs. This lung distribution was reduced in local delivery to the ileum and was lost in the oral gavage.

Table 9 summarizes the distribution of the 2 μm microspheres in both localized administrations, i.e. local delivery to the jejunum via injection and local delivery to the ileum via injection.

TABLE 9
Location comparison for 2 μm microspheres

| | Jejunum | | Ileum | |
|---|---|---|---|---|
| Tissue | % of Dose | % of Uptake | % of Dose | % of Uptake |
| Brain | 0.00 ± 0.00 | 0.00 ± 0.00 | 2.09 ± 2.09 | 22.27 ± 22.27 |
| Central | 0.09 ± 0.09 | 0.46 ± 0.46 | 1.17 ± 0.71 | 22.31 ± 11.89 |
| blood | | | | |
| Heart | 0.87 ± 0.54 | 3.74 ± 2.26 | 0.12 ± 0.08 | 3.20 ± 2.72 |
| Kidneys | 2.82 ± 1.03 | 12.86 ± 4.64 | 0.11 ± 0.10 | 1.80 ± 1.51 |
| Liver | 11.97 ± 4.06 | 54.70 ± 18.58 | 2.12 ± 1.23 | 39.01 ± 18.75 |
| Lungs | 0.31 ± 0.25 | 1.54 ± 1.26 | 0.51 ± 0.34 | 7.13 ± 5.24 |
| Portal | 0.24 ± 0.20 | 25.84 ± 24.73 | 0.12 ± 0.12 | 1.82 ± 1.78 |
| blood | | | | |
| Spleen | 0.20 ± 0.16 | 0.86 ± 0.67 | 0.16 ± 0.16 | 2.44 ± 2.44 |

| | | |
|---|---|---|
| Mass | 97.05 ± 7.86 | 105.33 ± 1.13 |
| Balance | | |
| Total | 16.03 ± 5.46 | 6.04 ± 1.19 |
| uptake | | |

As shown by the data in Table 9, following local delivery to both the jejunum and ileum the 2 μm microspheres distributed mostly to the liver. In the jejunum, there was also a statistically significant distribution to the kidneys. However, the total uptake in both regions was low, and the biodistribution results were based on small amounts of PS microspheres.

Table 10 summarizes the distribution of the 5 μm microspheres in both localized administrations, i.e. local delivery to the jejunum via injection and local delivery to the ileum via injection.

TABLE 10
Location comparison for 5 μm microspheres

| | Jejunum | | Ileum | |
|---|---|---|---|---|
| Tissue | % of Dose | % of Uptake | % of Dose | % of Uptake |
| Brain | 0.00 ± 0.00 | 0.00 ± 0.00 | 0.00 ± 0.00 | 0.00 ± 0.00 |
| Central | 0.17 ± 0.09 | 1.54 ± 1.13 | 0.11 ± 0.08 | 0.56 ± 0.33 |
| blood | | | | |
| Heart | 0.28 ± 0.26 | 3.21 ± 3.14 | 0.30 ± 0.18 | 2.22 ± 1.39 |
| Kidneys | 0.19 ± 0.19 | 2.31 ± 2.31 | 3.98 ± | 38.40 ± 9.90* |
| | | | 0.96+ | |
| Liver | 15.46 ± 5.59 | 75.87 ± 15.42 | 7.80 ± 4.02 | 55.08 ± 13.31 |
| Lungs | 0.81 ± 0.60 | 8.15 ± 7.35 | 0.21 ± 0.21 | 2.75 ± 2.75 |
| Portal | 0.97 ± 0.60 | 5.51 ± 2.38 | 0.12 ± 0.07 | 0.99 ± 0.72 |
| blood | | | | |
| Spleen | 0.43 ± 0.22 | 3.42 ± 2.16 | 0.00 ± 0.00 | 0.00 ± 0.00 |

| | | |
|---|---|---|
| Mass | 92.39 ± 6.06 | 93.79 ± 3.02 |
| Balance | | |
| Total | 19.40 ± 4.72 | 13.39 ± 5.27 |
| uptake | | |

As shown by the data in Table 10, following local delivery to both the jejunum and ileum, the 5 μm microspheres distributed mostly to the liver. In the ileum, a statistically significant amount of microspheres was detected in the kidneys. As with the 2 μm microspheres discussed above, the amount of total uptake was relatively low; and the biodistribution results are based on small amounts of PS microspheres.

Summary

These data indicate both a size-dependant and location-dependant uptake and biodistribution of microsphere systems. The size-dependence of uptake in the jejunum and ileum differ (see Tables 4 and 5) and indicate that different uptake mechanisms are taking place in the two regions. Further, this difference may account for the difference seen in biodistribution profiles of microspheres of varying size. Finally, this method of investigation has proven to be reliable and accurate.

Increasing the number of animals showed reproducibility and changed some of the general trends observed in Example 1. In all cases, the percentage of total uptake is significantly different. The biodistribution trends were also different. In Example 1, local delivery yielded distribution of the 0.5 μm microspheres to the liver only in the jejunum and not the ileum. After repeating the studies this trend was found to be moot; most of the distribution went to the liver in both cases. For 1 μm microspheres in Example 1, there was little distribution differences for local delivery the two regions. After repeating the experiments a new trend emerged in which the distribution shifted to the lung when delivered in the jejunum.

In addition to increasing the number of animals in each study group, more study groups were tested. The following additional groups were tested: 0.5 μm delivered with oral gavage, 1 μm delivered locally to the ileum, 2 μm delivered locally to the ileum, and 5 μm delivered locally to the ileum and jejunum. The results stemming from this data show an upper size limit for uptake, and also demonstrate a difference in the way these size limits occur in the two regions. In the jejunum the amount of uptake decreased with each increasing size (step-wise), but in the ileum the uptake is nearly the same for 0.5 and 1 um microspheres and then suddenly drops to a low amount for both the 2 and 5 μm microspheres. The ileum acts like a filter, excluding everything above 1 μm). This indicates that different mechanisms of uptake are taking place in each region, which may be the reason for the differences in biodistribution profiles.

### Example 3

### Biodistribution of Micro and Nanoparticles Containing Palladium (II) (Pd)

Particles containing palladium (II) acetate were formulated using the following protocol:
(1) Weigh out 100.0 mg of poly(adipic anhydride) (p[AA]) and 100 mg of palladium (II) acetate into a 20 ml glass scintillation vial. The theoretical loading of palladium (II) acetate is 50% and the theoretical loading of palladium (II) is approximately 23%;
(2) Mix 1000 mL of pentane and 6 mg of lecithin in a 1-L boiling flask using a magnetic stirring rod (600-800 RPM) for 15 minutes and keep covered;
(3) With ten (10) minutes remaining, add 10 mL of dichloromethane to (1);
(4) Bath sonicate at high resonance frequency for 120 seconds;
(5) Vortex on maximum speed for 30 seconds;
(6) Draw up (3) using a borosilicate glass syringe with luer-lock, injection needle;
(7) Keep needle tip 1.5 inches above the pentane-lecithin bath;
(8) Inject into pentane-lecithin bath by pouring the polymer-palladium solution into the glass syringe and allowing gravity to naturally inject the solution into the pentane-lecithin bath;
(9) Let sit for 45 seconds-1 minute;
(10) Immediately filter using a 0.1 micrometer Durapore (Millipore) filter; and
(11) Lyophilize for 48 hours and calculate percent yield.

This protocol produced p[AA]-Pd particles that contain a homogeneous distribution of palladium throughout the entire population of microspheres. The actual loading of palladium (as determined by ICPMS) was between 2 and 4%. The mean volumetric particle size was between 450-500 nm and 90% of the particles had a volumetric average diameter of less than 600 nm.

Method for Inductively Coupled Plasma Mass Spectrophotometry (ICPMS). Quantification of Palladium

The following method was used to quantitatively analyze the biodistribution of palladium nanospheres in specific rat fluids and tissues. Male Sprague-Dawley rats, weighing 175-200 g, were used throughout the study. Rats were fed standard rat feed and water ad libidum from time of arrival to 18 hours before the time of study. At that time, animals were only allowed water ad libidum. Animals were restricted access to food to allow for clearance of the duodenum and proximal jejunum. Study animals were first anesthetized with isoflurane and maintained under anesthesia peri-operatively. A 6-7 cm midline abdominal incision was made to expose intestines. Small intestine regions were identified using the ligament of Trietz (proximal jejunum) and increased Peyer's patch content (distal jejunum) as markers. A 6 cm section of the desired intestinal region was selected and gently cleared of its continents. The section was then ligated with 4-0 silk sutures by threading the suture through the intestinal mesentery (away from blood vessels) and then tying off both ends of the section taking care not to occlude blood flow.

A 1 mL suspension of p[AA]/Pd nanospheres (50 mg/ml) was injected with a 25 gauge needle into the section of interest. In the case of the control animal, 1 mL of saline was administered. Upon removal of the needle, a cauterizer was used prevent leakage of the nanosphere suspension. While maintaining anesthesia, rats were kept for five hours to allow for the uptake of microspheres. The lesion was sutured using 4-0 vicryl sutures during this period to avoid excessive loss of body heat and moisture.

Following the five hour period, the lesion was re-opened and extended into the thoracic cavity to expose the entire tissue cavity. A 5 ml central blood sample was obtained from the left ventricle. After obtaining the blood samples, all tissues are harvested. Order of removal is as follows; lungs, heart, spleen, kidneys, liver, intestinal section of interest and the brain. The intestinal section is thoroughly rinsed with saline and kept for processing with tissues (intestinal content). Fluids and tissue samples are kept at −20° C. until further analysis.

The organs are then digested using a combination of nitric acid and hydrochloric acid and then analyzed for palladium using inductively coupled plasma mass spectrophotometry (ICPMS).

Results=of Palladium (II) Acetate Quantification

The experiments in the proximal jejunum were conducted in a 1.5 inch length of the alimentary canal. The formulation contained 50 mg of nanospheres suspended in 1 ml of suspension media (0.5% SLS/1.0% PVP in PBS) and was incubated within the loop for five hours. Losses were estimated to be on the order of 5%. The results are very reproducible. The one exception involves samples contained within the isolated loop. This could be due to a leak in the ligature or losses of the sample during tissue processing. For complete results, see Table 11.

TABLE 11
Proximal Jejunum (n = 2)

| | | | Palladium | | Palladium | | |
|---|---|---|---|---|---|---|---|
| | | | detected in | | detected | Total Palladium | |
| | Raw Organ | | organ (μg | | in organ | detected in organ | |
| | Weight (g) | | Pd/g) | | (μg Pd/g) | (μg Pd) | |
| Organ | Rat 1 | Rat 2 | Rat 1 | Rat 2 | Average ± 1 SD | Rat 1 | Rat 2 |
| R. Lung | 1.1548 | 0.5233 | 0.14 | 0.143 | 0.1415 ± 0.0021 | 0.188966 | 0.088757 |
| L. Lung | 0.431 | 0.1873 | 0.122 | 0.153 | 0.1375 ± 0.0219 | 0.0629 | 0.036915 |
| Heart | 0.9739 | 0.9546 | 0.091 | 0.087 | 0.089 ± 0.0028 | 0.098451 | 0.0894 |
| Liver | 8.5204 | 7.0019 | 0.372 | 0.247 | 0.3095 ± 0.0884 | 3.19592 | 1.752014 |
| Spleen | 0.6991 | 0.5398 | 0.342 | 0.149 | 0.2455 ± 0.1365 | 0.261949 | 0.099624 |
| R. | 1.1967 | 0.8915 | 3.04 | 3.69 | 3.365 ± 0.4596 | 3.823605 | 3.483442 |
| Kidney | | | | | | | |
| L. | 1.119 | 0.9228 | 3.18 | 3.25 | 3.215 ± 0.0495 | 3.715625 | 3.116281 |
| Kidney | | | | | | | |
| Brain | 1.9298 | 1.7593 | 0.06 | 0 | 0.03 ± 0.0424 | 0.120992 | 0 |
| Isolated | 3.3522 | 2.8824 | 212 | 458 | 335 ± 173.9483 | 735.5489 | 1436.78 |
| Loop | | | | | | | |
| Central | 7.2902 | 4.7893 | 0.143 | 0.142 | 0.1424 ± 0.0007 | 1.136911 | 0.763487 |
| Blood | | | | | | | |
| Residual | 2.3414 | 3.4904 | 1.51 | 0.196 | 0.853 ± 0.9291 | 4.076854 | 0.752173 |
| Blood | | | | | | | |

| | | |
|---|---|---|
| Total μg Pd detected | 752.2311 | 1446.962 |
| Total μg Pd administered | 1600 | 1600 |
| Detection Efficiency | 0.470144 | 0.904352 |

### Example 4

### Mucoadhesion of Metals Compared with Non-Adhesive Materials and Mucoadhesive Polymers Tested In Vitro

To determine the relative bioadhesive forces for metal surfaces, a representative metal sample, a magnetic stainless steel sample was tested. Stainless steel samples were tested for bioadhesion on the small intestine of rats and pigs, as well as on artificial substrates containing mucin, the main glycoprotein component of mucus.

Materials and Methods

Small Intestine

Rat and pig small intestine was harvested after the animal was euthanized. A midline incision was made and the small intestine was isolated. The area of interest was clamped and excised. The lumen was then washed with phosphate buffered saline (PBS) to remove any visible chyme. The tissue was then stored at −20° C.

The tissue was allowed to equilibrate to room temperature for 30 minutes before testing. At this time, a cut in the tissue was made longitudinally and rinsed with PBS once more to remove any loose mucus. The tissue was then placed in a cell to hold it down, and it was bathed in an excess of PBS.

Mucin/Agarose Gels

Mucin/Agarose gels were used as an artificial substrate in lieu of the gastrointestinal tract. Mucin (Type II, Sigma-Aldrich, St. Louis, Mo.) was added at 0, 4, 7, and 10% w/v to stirring distilled water. Once in solution, the temperature of the water was increased approximately 10° C. over the gelling temperature of agarose. At this time, agarose (high gelling temperature, Fluka, St. Louis Mo.) was added at 4% w/v. After dissolving, the solution was poured into containers. The gels were allowed to form by cooling to room temperature. Then the gels were stored at 4° C.

Texture Analyser

The TA.XT Plus Texture Analyser (Texture Technologies, Scarsdale, N.Y.) was used to measure bioadhesion. Two different types of tests were performed. The first, an adhesive test, was performed by lowering the probe of interest to the substrate. When a specified target force was reached, this force was maintained by changing the height of the probe. Once a specified period of time had passed, the probe was lifted away from the substrate.

The second test was a hold distance test. The probe was again lowered to the substrate. When the target force was reached, the probe stopped and maintained the distance. After a specified period of time, the probe was removed from the substrate.

To measure bioadhesion, two measurements were used, i.e. fracture strength and tensile work. Fracture strength was obtained by dividing the peak load (or maximum force) by the projected surface area (PSA). PSA was approximated in these calculations as the largest cross-sectional area of the probe. The tensile work is the work needed to separate the probe from the substrate. It was calculated by the area under the curve of the positive portion of the tensile curve.

Statistical Analysis

SPSS Statistical Software (version 11.5, Chicago, Ill.) was used to run analysis of variance (ANOVA) and the Tukey Honestly Significant Difference (HSD) post-hoc test. A p value of less than 0.05 is deemed statistically significant.

Results

Small Intestine

Using the adhesive test described above, with a target force of 30 g, on pig tissue kept at 37° C., the measured fracture strength of 14 runs was 5198 mN/cm² with a standard deviation of 726. Tensile work was determined to be 17852 nJ with a standard deviation of 5111.

Using the adhesive test described above, with a target force of 30 g, on rat tissue kept at 37° C., the measured fracture strength of 17 runs was 5905 mN/cm² with a standard deviation of 3089. Tensile work was determined to be 4169 nJ with a standard deviation of 7352.

Using the hold distance test described above, with a target force of 30 g, on pig tissue kept at 37° C., the measured fracture strength of 18 runs was 1949 mN/cm² with a standard deviation of 2667. Tensile work was determined to be 2082 nJ with a standard deviation of 2245.

Mucin/Agarose Gels

Stainless steel was compared to the following non-adhesive materials and adhesive polymers: two waxes (candelilla and carnauba), poly(caprolactone), and poly(fumaric-co-sebacic acid). All probes were of fairly comparable sizes, with projected surface areas of approximately 2-7 mm² The measured fracture strength and tensile work with standard deviations for the different materials in the mucin/agarose gels is provided in Tables 12, 13, 14, and 15.

TABLE 12
Materials on 0% Mucin

| | | Fracture Strength | Tensile Work |
|---|---|---|---|
| Probe | n | (mN/cm²) | (nJ) |
| Candelilla Wax | 6 | 98 ± 49 | 393 ± 110 |
| Carnauba Wax | 6 | 102 ± 45 | 377 ± 235 |
| PCL | 6 | 172 ± 71 | 413 ± 88 |
| FASA | 6 | 109 ± 23 | 355 ± 63 |
| Stainless Steel | 6 | 405 ± 70 | 388 ± 127 |

TABLE 13
Materials on 4% Mucin

| | | Fracture Strength | Tensile Work |
|---|---|---|---|
| Probe | n | (mN/cm²) | (nJ) |
| Candelilla Wax | 6 | 162 ± 44 | 170 ± 27 |
| Carnauba Wax | 6 | 187 ± 36 | 168 ± 43 |
| PCL | 10 | 423 ± 179 | 341 ± 176 |
| FASA | 13 | 368 ± 255 | 608 ± 320 |
| Stainless Steel | 10 | 671 ± 475 | 302 ± 248 |

TABLE 14
Materials on 7% Mucin

| | | Fracture Strength | Tensile Work |
|---|---|---|---|
| Probe | n | (mN/cm²) | (nJ) |
| Candelilla Wax | 6 | 327 ± 92 | 2140 ± 1003 |
| Carnauba Wax | 12 | 301 ± 71 | 340 ± 93 |
| PCL | 6 | 635 ± 133 | 1170 ± 376 |
| FASA | 12 | 461 ± 115 | 350 ± 115 |
| Stainless Steel | 6 | 1128 ± 195 | 580 ± 169 |

TABLE 15
Materials on 10% Mucin

| | | Fracture Strength | Tensile Work |
|---|---|---|---|
| Probe | n | (mN/cm²) | (nJ) |
| Candelilla Wax | 10 | 331 ± 79 | 2002 ± 826 |
| Carnauba Wax | 7 | 275 ± 92 | 2155 ± 505 |
| PCL | 6 | 302 ± 116 | 1802 ± 919 |
| FASA | 6 | 700 ± 75 | 1809 ± 271 |
| Stainless Steel | 6 | 1250 ± 184 | 1010 ± 129 |

Using 4, 7, and 10% mucin agarose gels, stainless steel showed a statistically significant difference in fracture strength from the waxes at 4% mucin, and all materials at 7 and 10% mucin.

Stainless steel only showed a significant difference on the 10% mucin agarose gels with respect to carnauba wax for tensile work.

### Example 5

### Mucoadhesion of Iron Microparticles Tested In Vivo

A 350 g Sprague-Dawley rat was orally gavaged with one milliliter of an iron microparticles <10 microns (99.9+% Aldrich) 50 wt % suspension in distilled water. X-ray radiography was performed to track the gastrointestinal (GI) transit time. Two hours after dosing some of the dosage has entered the small intestine. However, the majority of the dosage remained in the stomach for at least eight hours, and only a small portion of the dosage was excreted within eight hours. Twenty-two hours after dosing, the majority of the dosage has been excreted and a small portion remained in the greater curvature of the stomach. The prolonged transit time of the iron microparticles demonstrates their mucoadhesion in vivo.

### Example 6

### Mucoadhesion of Iron Microparticles After Application of Magnetic Field Tested In Vitro

Explanted porcine small intestinal tissue was situated in an acrylic tissue holder so that the mucus-lined lumen was open to atmospheric conditions. Iron microparticles <10 microns (99.9+% Aldrich) 50 wt % suspension in distilled water were pipetted onto two separate sections of the tissue sample. Beneath one portion of the tissue sample, a neodymium-iron-boron (NIB) grade N38 rod magnet (½″ diameter×1″ length K&J Magnetics, Inc.) was placed to magnetically attract one population of iron microparticles into the mucus layer. The other population of iron microparticles was not exposed to the magnetic field. After allowing settling for five minutes, the tissue sample was removed from the magnet and both sections of the tissue sample were washed under streaming water.

The population of iron microparticles that had not been exposed to a high strength magnetic field rinsed away immediately. In contrast, a large portion of the population of iron microparticles that had been exposed to the high strength magnetic field remained embedded within the mucosa after 60 seconds of rinsing. These results indicate that microspheres containing magnetic material can be imbedded in the mucosa of intestinal tissue by applying an extracolporeal magnet for a suitable period of time. Additionally, these results indicate that the mucoadhesive property of a metal, such as iron and/or its oxides, will maintain the adherence of the dosage formulation to the tissue site, even after the magnetic field has been removed.

### Example 7

### Localization Iron Microparticles and Nickel-Coated Nib Rod Magnet Due to the Application Magnetic Field Tested In Vivo

A. Site-Directed Delivery of Iron Microparticles

A 350 g Sprague-Dawley rat was orally gavaged with one milliliter of an iron powder <10 microns (99.9+% Aldrich) 33 wt % suspension in distilled water. Within one hour, while the dosage was within the stomach, an extracorporeal neodymium-iron-boron (NIB) grade N38 rod magnet (½″ diameter×1″ length K&J Magnetics, Inc.) was brought into contact with the ventral abdominal skin of the rat. Magnetic iron particles aligned with the magnetic field lines of the magnet within the lumen of the stomach as evidenced by x-ray radiography. During the application of the extracorporeal magnet, the microparticles were localized within the stomach and their spatial orientation within the lumen changed from randomly distributed to ordered along magnetic field lines at the stomach wall.

These results demonstrate that ferromagnetic material micron-sized-dosage-forms can be localized within the gastrointestinal (GI) tracts of mammals by the application of an extracorporeal magnet.

B. Site-Directed Delivery of Nickel-Coated NIB Rod Magnet in Gelatin Capsule

A nickel-coated NIB rod magnet ( 1/16″ diameter× 1/16″ length K&J Magnetics, Inc.) manually loaded into a size 9 gelatin capsule (Torpac, Inc.) was delivered to a 350 g Sprague-Dawley rat by oral gavage. An extracorporeal ring magnet (½″ OD×¼″ ID×¾″ length K&J Magnetics, Inc.) was tied to the ventral abdomen of the rat with ⅛″ diameter poly(dimethyl siloxane) tubing. The ingested rod magnet was localized to the anatomic space that most closely apposed the ingested magnet to the extracorporeal magnet aligned in the direction of magnetic polarity. The ingested magnet was retained within the stomach at the site of application of the extracorporeal magnet for 24 hours while the rat had ad libitum access to food and water.

These results demonstrate that magnetic macro-dosage-forms can be localized within the GI tracts of mammals by the application of an extracorporeal magnet.

### Example 8

### Localization of Gelatin Capsule Containing Nickel-Coated NIB Magnet Due to the Application Magnetic Field and Simulation of Release of Active Agent In Vivo

A nickel-coated NIB rod magnet ( 1/16″ diameter× 1/16″ length K&J Magnetics, Inc.) manually loaded into a size 9 gelatin capsule (Torpac, Inc.) and the void space was packed with the radioopaque marker, barium sulfate powder (98.1% Malinckrodt, Inc.). A Sprague-Dawley rat was gavaged with the dosage form and an extracorporeal neodymium-iron-boron (NIB) grade N38 rod magnet (½″ diameter×1″ length K&J Magnetics, Inc.) was brought into close proximity with the ventral-lateral abdomen and then removed. X-ray radiography shows the ingested magnet distinctly separate from the remainder of the capsule in the stomach of the rat. Barium sulfate was observed diffusing into the stomach through the exit hole in the gelatin capsule using X-ray radiography.

These results demonstrate that an extracorporeal magnet can be used to trigger the release of a model powder in a localized fashion within the GI tracts of mammals.

Those skilled in the art will recognize, or be able to ascertain using no more than routine experimentation, many equivalents to the specific embodiments of the invention described herein. Such equivalents are intended to be encompassed by the following claims.

1. An oral dosage formulation for enhanced relative delivery of a therapeutic, prophylactic or diagnostic agent to the lymphatic capillaries comprising
microparticles containing the therapeutic, prophylactic or diagnostic agent to be delivered, in the form of a homogenous population wherein preferably 75% of the microparticles have a mean diameter of about 1 or 0.5 micron.

2. (canceled)

3. The formulation of claim 1, further comprising a metallomucoadhesive or magnetic material.

4. The formulation of claim 3, wherein the metallomucoadhesive material is selected from the group consisting of chromium and its oxides, iron and its oxides, titanium and its oxides, aluminum and its oxides, nickel and its oxides, zinc and its oxides, neodymium and its oxides, gold, silver and its oxides and salts, copper and its oxides, and alloys thereof.

5. The formulation of claim 3, wherein the magnetic material is a ferromagnetic or superparamagnetic compound.

6. The formulation of claim 5, wherein the magnetic material is selected from the group consisting of martensitic stainless steels, iron oxides, neodymium iron boron ceramic, alnico (AlNiCo), and samarium cobalt.

7. The formulation of claim 1, wherein the formulation is a solid oral dosage formulation.

8. The formulation of claim 1, wherein the formulation further comprises an enteric coating.

9. A method for enhanced relative delivery of a therapeutic, prophylactic or diagnostic agent to the lymphatic system comprising
orally administering to a patient in need thereof the formulation of claim 1 to the ileum.

10. The method of claim 9, further comprising applying an extracorporeal magnet to the outside surface of the patient's body at a site in an area that apposes the ileum.

11. The method of claim 10, further comprising removing the extracorporeal magnet from the site after a period of time effective to attach the formulation to the ileum via mucoadhesive forces.

12. The method of claim 11, wherein the period of time ranges from 1 to 8 hours.

13. The method of claim 10, further comprising removing the extracorporeal magnet from the site after a period of time effective to attach the formulation to the ileum via Mucoadhesive forces release.

14. The method of claim 12, wherein the period of time ranges from 5 minutes to 24 hours.

15. A method for enhanced relative delivery of a therapeutic, prophylactic or diagnostic agent to the portal circulation and liver comprising
orally administering to a patient in need thereof the formulation of 2 to the jejenum.

16. The method of claim 15, further comprising applying an extracorporeal magnet to the outside surface of the patient's body at a site in an area that apposes the jejunum.

17. The method of claim 16, further comprising removing the extracorporeal magnet from the site after a period of time effective to attach the formulation to the jejunum via mucoadhesive forces.

18. The method of claim 17, wherein the period of time ranges from 1 to 8 hours.

19. The method of claim 16, further comprising removing the extracorporeal magnet from the site after a period of time effective to release the therapeutic, prophylactic or diagnostic agent from the formulation.

20. The method of claim 19, wherein the period of time ranges from 5 minutes to 24 hours.

21. An oral dosage formulation for enhanced relative delivery of a therapeutic, prophylactic or diagnostic agent to a site within the gastrointestinal tract comprising
the therapeutic, prophylactic or diagnostic agent to be delivered and a magnetic material.

22. The formulation of claim 21, wherein the formulation is a solid oral dosage formulation in a form selected from the group consisting of tablets, capsules, and osmotic-pump-based delivery systems.

23. A kit comprising an oral dosage formulation of claim 1 and an extracorporeal magnet.

24. A method for enhanced uptake of a therapeutic, prophylactic or diagnostic agent to a site in the gastrointestinal tract comprising
selecting a site in the gastrointestinal tract for delivery of the therapeutic, prophylactic or diagnostic agent,
orally administering to a patient in need thereof the formulation of claim 21, and
applying an extracorporeal magnet to the outside surface of the patient's body at a second site in an area that apposes the selected site in the gastrointestinal tract.

25. The method of claim 24, further comprising removing the extracorporeal magnet from the second site after a period of time effective to attach the formulation to the selected site via mucoadhesive forces.

26. The method of claim 25, wherein the period of time ranges from 1 to 8 hours.

27. The method of claim 24, further comprising removing the extracorporeal magnet from the second site after a period of time effective to release the therapeutic, prophylactic or diagnostic agent from the formulation.

28. The method of claim 26, wherein the period of time ranges from 5 minutes to 24 hours.
